(19) United States
(12) Patent Application Publication
(10) Pub. No.: US 2022/0206002 A1
(43) Pub. Date: Jun. 30, 2022

(54) SYSTEMS AND METHODS FOR DETECTING SUBSTANCES IN A FLUID USING TAPERED FIBERS

(71) Applicant: Uttam K. Sinha, San Marino, CA (US)

(72) Inventor: Uttam K. Sinha, San Marino, CA (US)

(21) Appl. No.: 17/556,957

(22) Filed: Dec. 20, 2021

Related U.S. Application Data
(60) Provisional application No. 63/132,456, filed on Dec. 30, 2020.

(51) Int. Cl. G01N 33/569; G01N 33/50; G01N 21/77

(52) U.S. Cl. CPC G01N 33/56994; G01N 33/5091; G01N 2201/06113; G01N 2333/05; G01N 2201/08; G01N 21/7703

(57) ABSTRACT

Systems and methods for detecting a substance in a fluid and detecting a condition based on the substance in the fluid are described herein. Electromagnetic radiation travels through tapered fibers while substance is bound to the tapered fibers. The phase change of the electromagnetic radiation caused by the bound substance is used to detect the substance in the fluid and to predict the likelihood of a condition based on the bound substance.

### CROSS-REFERENCE TO RELATED APPLICATIONS

This application claims priority from U.S. Provisional Application No. 63/132,456, which is incorporated herein by reference in its entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### FIELD

The present teachings relate to systems and methods for detecting substances in a fluid.

### INTRODUCTION

Diagnosing medical or physiological conditions can be a laborious affair. Current techniques for diagnosing such conditions include lateral flow assay, ELISA, HPLC, cell sorting, and so forth. However, these examples have significant drawbacks, one of which is the need for bulky and expensive equipment. A system for detecting a substance in a fluid without such drawbacks is needed. Indeed, developing a simple and repeatable way to do so would be of great benefit to a number of areas, such as healthcare, food security and quality, and environmental contamination.

### SUMMARY

The present teachings include a system for detecting a substance in a fluid. The system is comprised of an electromagnetic radiation (EMR) source, an input fiber, at least one tapered fiber, an output fiber, and a photodetector. The tapered fiber, in one embodiment, may be a single fiber. In another embodiment, there may be several tapered fibers. The taper is made by locally heating the fiber close to (but below) the glass transition temperature and then stretching the fiber ends in a controlled manner to produce a waist region. In an embodiment, the diameter of the waist for a tapered fiber may be between 3 microns and 8 microns. In another embodiment, the diameter of the waist for a tapered fiber may be between 8 microns and 14 microns. In another embodiment, the diameter of the waist for a tapered fiber may be between 3 microns and 14 microns. The input fiber connects at one end to the electromagnetic radiation source and at the other end to the tapered fiber. The substance being analyzed exists in a fluid (liquid or gas/vapor) is bound to the surface of the tapered fiber. The output fiber connects at one end to the tapered fiber and at the other end to the photodetector. In one embodiment, the electromagnetic radiation source and photodetector exist in the same machine. In another embodiment, the electromagnetic radiation source and photodetector are separate machines. In an embodiment, the system may be used right away to collect data. In another embodiment, the system may need some time to warm up to collect data. In such instances, up to 2 hours warm up time may be needed.

In accordance with a further aspect, the electromagnetic radiation source may exist in several embodiments. In one embodiment, the electromagnetic radiation source is a laser-emitting diode. In another embodiment, the EMR source is a laser diode. In another embodiment, the EMR source is a pulse wave laser. In another embodiment, the EMR source is a continuous wave laser. In yet another embodiment, the EMR source is a tunable laser. All other EMR sources known in the art are suitable for providing the electromagnetic radiation that travels through the tapered fiber.

In accordance with yet another aspect, an auto-injector pushes substance-containing fluid to the vicinity of the tapered fiber for the substance to bind to the tapered fiber. While an auto-injector performs the function of moving substance-containing fluid to the tapered fiber for substance to bind to the tapered fiber, other ways of binding substance to the tapered fiber are possible, using phenomenon such as force, pressure, kinetic energy, and other means of delivering the substance to the tapered fiber's surface.

In accordance with yet another aspect, regardless of the configuration (single tapered fiber or multiple tapered fibers), the fibers are housed in a cell. The substance-containing fluid flows through the cell, with the substance binding to the tapered fiber within the cell. The cell may be constructed from a number of materials, including polymers, ceramics, and metals. In an embodiment, the cell is constructed from Teflon.

In accordance with yet another aspect, the substance may exist in many forms. Examples of substances that may bind to the surface of the taper fiber include bacteria, viruses, parasites, hormones, electrolytes, biomolecules, biomarkers, analytes, fungus, proteins, nucleotides, volatile organic compounds, and organic molecules. Essentially, any substance that can bind to the tapered fiber may qualify for the system.

In accordance with yet another aspect, the fluid that the substance exists in may take on various forms, both liquid, gas, and vapors. Examples of suitable fluids for the substance include cerebrospinal fluid, drain fluid, saliva, urine, blood, plasma, tears, bronchioalveolar lavage fluid, serum, pleural fluid, synovial fluid, peritoneal fluid, amniotic fluid, gastric fluid, lymph fluid, interstitial fluid, tissue homogenate, cell extracts, sputum, stool, physiological secretions, mucus, sweat, milk, semen, seminal fluid, vaginal secretions, fluid from ulcers and surface eruptions, blisters, and abscesses, forever chemicals in water, perfluorooctanoic acid in water, perfluorooctanesulfonic acid in water, and extracts of tissues including biopsies of normal, malignant, and suspect tissues, and exhaled breath condensate.

In accordance with yet another aspect, the tapered fiber binds more than one substance. In one embodiment, multiple tapered fibers may bind multiple substances. With each tapered fiber housed in the same cell, each tapered fiber in this arrangement may bind a different substance. In this embodiment, the binding of the various substances changes the refractive index of the fibers, which causes a phase shift of the electromagnetic radiation.

In accordance with yet another aspect, a chemically active moiety is bound to the surface of the tapered fiber. The chemically active moiety assists in binding the substance to the tapered fiber.

In accordance with yet another aspect, a molecular sensing element binds to the chemical active moiety. The substance binds to the molecular sensing element.

In accordance with yet another aspect, the chemically active moiety may be selected from many types. In one embodiment, amino silanes are the chemically active moiety. In another embodiment, alkoxy silanes are the chemically active moiety. Other embodiments may use one of epoxy silanes, vinyl silanes, methacryloxy silanes, isocyanato silanes, mercapto silanes, polysulfide silanes, ureido silanes, chromium, orthosilicate, inorganic ester, titanium, and zirconium systems. They are all chemically active moieties that may readily bind to the tapered fiber via hydrolysis and condensation reactions.

In accordance with yet another aspect, the molecular sensing element may be chosen from many types of binding molecules. In one embodiment, aptamers are the molecular sensing element. In another embodiment, antibodies are the molecular sensing element. In yet another embodiment antibody fragments are the molecular sensing element. Any element that can bind to the chemically active moiety is suitable. In an embodiment, immunoglobulin G antibodies may be the molecular sensing element.

The present teaching includes a method for detecting a substance, comprising binding the substance to the tapered fiber, passing electromagnetic radiation through the tapered fiber from an EMR source, and obtaining a value from a photodetector. The value from the photodetector is affected by the electromagnetic radiation's phase shift caused by traveling through the tapered fiber, as well as the substance bound to the tapered fiber surface. As the concentration of the substance in the fluid changes, the phase shift similarly changes, as does the value.

In accordance with a further aspect, the substance exists in a fluid.

In accordance with yet another aspect, the tapered fiber or tapered fibers are housed in a cell, with the electromagnetic radiation passing through the tapered fibers or tapered fibers, and the substance-containing fluid passing through the cell, allowing the substance to bind to the tapered fiber surface via the molecular sensing element. The molecular sensing element binds to the chemically active moiety. The chemically active moiety binds to the tapered fiber.

In accordance with yet another aspect, the EMR source, in one embodiment, is a light-emitting diode. In another embodiment, the EMR source is a laser diode. Other embodiments for the EMR source include pulse wave laser, continuous wave laser, and tunable laser.

The present teachings also include a method for detecting a condition, comprising binding a substance to the tapered fiber, passing electromagnetic radiation through the tapered fiber from a EMR source, generative a value based on the substance bound to the tapered fiber and the phase shift experienced in the tapered fiber by the electromagnetic radiation. The value is depicted as a score. The score signifies the likelihood of having the condition.

In accordance with a further aspect, the likelihood of having a condition is dependent on the score's value from zero. Closer to zero, there is less likelihood of the condition. Further from zero, there is more likelihood of the condition. In another embodiment, the opposite is the case: the closer to zero, there is more likelihood of the condition, and the further from zero, there is less likelihood of the condition. The score may be expressed in any suitable or useful level of granularity such as with discrete categories (e.g., no condition, condition, unknown), alphabetic score/grade, or other quantitative indicator.

In accordance with yet another aspect, the condition may be selected from a variety of conditions. In one embodiment, cancer is the condition being detected. In another embodiment, mononucleosis is the condition being detected. In other embodiments, other conditions are detected, including COVID-19, Anthrax, pregnancy, thyroid cancer, prostate cancer, lung cancer, esophageal cancer, congestive heart failure, myocardial infarction, diabetes, glucose anomalies, Parkinson's Disease, Alzheimer's Disease, Tuberculosis, and common cold. Essentially, any condition that is associated with a particular substance is detectable as long as that substance can bind to the tapered fiber. Conditions associated with certain physiologies are also detectable, including cardiovascular, neurological, and orthopedic.

In accordance with yet a further aspect, a medical professional may compare the score generated by the method to the medical professional's own assessment to determine false positives and false negatives. This may serve to compare the medical professional's assessment to the method to determine accuracy.

In accordance with yet a further aspect, the score may change based on the substance bound to the tapered fiber. As the amount of substance in the fluid changes, the score changes. Also, as the number of fibers changes, the score changes.

These and other features, aspects, and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1 is an embodiment of a tapered fiber system.

FIG. 2a is a depiction of a tapered fiber.

FIG. 2b is a depiction of the system with biomolecular pairs and organic molecules surrounding a functionalized surface

FIG. 3 is a depiction of an example graph where power is plotted against wavelength.

FIG. 4 is a depiction of an example graph where phase is plotted against time.

FIG. 5 is a depiction of an exemplary portable version of the system.

FIG. 6 is a depiction of multiple fibers arranged in a cell.

FIG. 7 is a depiction of a functionalized surface binding a chemically active moiety, which binds a molecular sensing element.

FIG. 8 is an exemplary perspective view of a cell.

FIG. 9 is an exemplary bottom view of a cell.

FIG. 10 is an immunoglobulin structure.

FIG. 11 is a schematic of IL-8.

FIG. 12 is a phase change diagram showing the effect of adding antigen to a cell.

FIGS. 13 a), 13 b), 13 c), and 13 d) are representative graphs of signals from cells with tapered fiber lengths of 4 mm, 6 mm, 8 mm, and 10 mm, respectively.

### DETAILED DESCRIPTION

Abbreviations and Definitions

To facilitate understanding of the invention, a number of terms and abbreviations as used herein are defined below as follows:

Substance: As used herein, the term “substance” refers to the material that binds to molecular sensing element, with the molecular sensing element binding to a chemically active moiety, and with the chemically active moiety binding to the tapered fiber. The substance is detectable by the system.

Binding Molecule: As used herein, the term “binding molecule” refers to an intact immunoglobulin including single-domain antibodies, including chimeric, or humanized monoclonal antibodies, or to an antigen-binding and/or variable domain comprising a fragment of an immunoglobulin that competes with the intact immunoglobulin for specific binding to the binding partner of the immunoglobulin. In one non-limiting example, the binding molecule can be an immunoglobulin capable of binding a coronavirus (e.g. SARS-CoV2). Regardless of structure, the antigen-binding fragment binds with the same antigen that is recognized by the intact immunoglobulin. An antigen-binding fragment can comprise a polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 35 contiguous amino acid residues, at least 40 contiguous amino acid residues, at least 50 contiguous amino acid residues, at least 60 contiguous amino acid residues, at least 70 contiguous amino acid residues, at least 80 contiguous amino acid residues, at least 90 contiguous amino acid residues, at least 100 contiguous amino acid residues, and at least 130 contiguous amino acid residues of the amino acid sequence of the binding molecule.

Antigen-binding fragments include complementarity determining region (CDR, and particularly CDR3) fragments, and polypeptides that contain at least a fragment of an immunoglobulin that is sufficient to confer specific antigen binding to the polypeptide. The above fragments may be produced synthetically or by enzymatic or chemical cleavage of intact immunoglobulins or they may be genetically engineered by recombinant DNA techniques. The methods of product are well known in the art and are described in for example, Antibodies: A Laboratory Manual, Edited by: E. Harlow and D. Lane (1988), Cold Spring Harbor Laboratory, Cold Spring Harbor, N.Y., which is incorporated herein by reference. A binding molecule or antigen-binding fragment thereof may have one or more binding sites. If there is more than one binding site, the binding sites may be identical to one another or they may be different.

The binding molecule can be a naked or unconjugated binding molecule but can also be part of an immunoconjugate. A naked or unconjugated binding molecule is intended to refer to a binding molecule that is not conjugated, operatively linked or otherwise physically or functionally associated with an effector moiety or tag, such as inter alia a toxic substance, a radioactive substance, a liposome, an enzyme. It will be understood that naked or unconjugated binding molecules do not exclude binding molecules that have been stabilized, multimerized, humanized or in any other way manipulated, other than by the attachment of an effector moiety or tag. Accordingly, all post-translationally modified naked and unconjugated binding molecules are included herein, including where the modifications are made in the natural binding molecule-producing cell environment, by a recombinant binding molecule-producing cell, and are introduced by the hand of man after initial binding molecule preparation. The term naked or unconjugated binding molecule does not exclude the ability of the binding molecule to form functional associations with effector cells and/or molecules after administration to the body, as some of such interactions are necessary in order to exert a biological effect. The lack of associated effector group or tag is, therefore, applied in definition to the naked or unconjugated binding molecule in vitro, not in vivo.

Electromagnetic radiation (EMR): As used herein, the term “electromagnetic radiation” refers to waves of the electromagnetic field propagating through space, to include radio waves, microwaves, visible light, ultraviolet, X-rays, gamma rays, laser light, and electromagnetic radiation from an electromagnetic radiation source.

Fluid: As used herein, the term “fluid” refers to a material that continuously deforms under an applied shear stress, including liquids and vapors.

Aptamers: As used herein, the term “aptamers” refer to short sequences of DNA or proteins binding to a chemically active moiety.

Antibodies: As used herein, the term “antibodies” are broadly defined to include: Y-shaped proteins composed of multiple amino acid chains joined by cysteine-cysteine disulfide bonds. Hypervariable regions at the tip of the antibody fragments have short amino acid sequences referred to as antigens that bind to specific antibodies.

Antigens: As used herein, the term “antigens” are broadly defined to include: foreign viruses, bacteria, proteins, cells, or non-foreign molecules that the body itself creates.

Systems and Methods for Detecting Substances in a Fluid Using Tapered Fibers

The present invention is directed to systems of detecting substances in a fluid and methods for detecting substances and detecting physiological conditions. The system is based on the phase shift of electromagnetic radiation traveling through a tapered fiber with the substance bound to the surface of the tapered fiber. Received data may be analyzed using innovated Fourier transformation method to find phase changes directly related to the molecules coated on tapered fiber. The evanescent electromagnetic (EM) field extends outside the tapered fiber, enabling detection of minute changes of the refractive index close to the surface of the fiber. The value caused by this phenomenon is depicted as a score, with the score signifying the likelihood of having a condition. Many substances are detectable by the system, just as long as the substance is bound to the tapered fiber. Examples of substances include biomarkers, biomolecules, bacteria, viruses, parasites, proteins, and nucleotides. The fiber binds a chemically active moiety, such as an amino silane, and the chemically active moiety binds a molecular sensing element, like an antibody. The substance binds to the molecular sensing element. Any condition associated with a substance that binds to the tapered fiber (e.g. Epstein-Barr Virus binding to the tapered fiber to detect mononucleosis) is detectable.

Advantages of the systems and methods herein include (i) label-free detection of substances; and (ii) self-testing with a fluid sample. While a system that handles substances in a liquid (e.g. saliva) are possible, systems that handles substances in vapor or gases are also available. Any fluid environment is possible. Nucleic acid assays (i.e. RT-PCR) require extraction of viral nucleic acid with trained personnel and specialized equipment. The system and methods herein eliminates nucleic acid extraction, while providing an accurate result in minutes instead of hours.

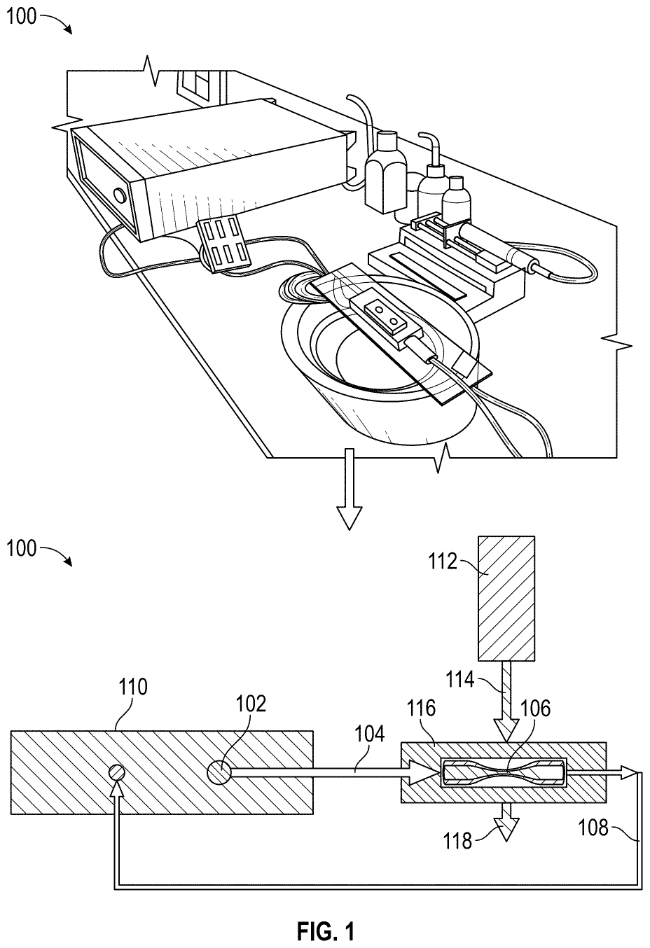

Referring to FIG. 1, an embodiment of the system 100 is depicted. In this embodiment, a liquid is the fluid delivering a substance to the tapered fiber 106. A substance-containing liquid is placed into injection tube 114. Injection tube 114 is connected to auto injector 112. Auto injector 112 may include a plunger or syringe system that forms a seal with injection tube 114. A pressurized environment is applied against the liquid to transport the substance to tapered fiber 106, housed in a cell 116. To relieve pressure and control the rate transport of the substance, a leak tube 118 is connected to cell 116.

The fluid contains substances that bind (i.e. covalently tether) to a surface of tapered fiber 106, which is a functionalized tapered fiber. The substances may take on many forms, such as biomolecules, viruses, bacteria, and parasites. The adhesion of the substances to the surface of tapered fiber 106 form a binding complex, which causes a phase-shift of electromagnetic radiation (EMR) transmitted through the fiber upon the EMR impinging the binding complex. The EMR may be emitted from an EMR source, such as fiber laser output 102 and transmitted through input fiber 104.

Input fiber 104 allows for EMR to reach the tapered fiber 106. The EMR (laser light in this embodiment) impinging the binding complex is subsequently transmitted through output fiber 108, which is connected to fiber photodetector 110. The light impinges the binding complex as to elicit a signal for processing by fiber photodetector 110. The signal (or value) generated is dependent on the phase shift of the light and the substance bound to the tapered fiber 106.

The system's detection capability is quite sensitive. In one example, the system can detect 10 picogram (pg)/milliliter (ml) of IL-8 (7.1×10⁵ IL-8 particles/μl). SARS-CoV-2 virions are approximately 120 nanometers (nm) in diameter. One of skill in the art would recognize, for example, that the system may detect from about 1 pg/ml of an interleukin or other polypeptide to about 1000 pg/ml of the interleukin or other polypeptide. In a non-limiting example, the system may detect from about 10 pg/ml of an interleukin or other polypeptide to about 900 pg/ml of interleukin or another polypeptide. In another non-limiting example, the system may detect from about 20 pg/ml of interleukin or other polypeptide to about 800 pg/ml of interleukin or other polypeptide. In another non-limiting example, the system may detect from about 30 pg/ml of interleukin or another polypeptide to about 700 pg/ml of interleukin or other polypeptide. In another non-limiting example, the system may detect from about 40 pg/ml of interleukin or other polypeptide to about 600 pg/ml of interleukin or other polypeptide. In another non-limiting example, the system may detect from about 50 pg/ml of interleukin or other polypeptide to about 500 pg/ml of interleukin or other polypeptide. In another non-limiting example, the system may detect from about 60 pg/ml of interleukin or other polypeptide to about 400 pg/ml of interleukin or other polypeptide. In another non-limiting example, the system may detect from about 70 pg/ml of interleukin or other polypeptide to about 300 pg/ml of interleukin or other polypeptide. In another non-limiting example, the system may detect from about 80 pg/ml of interleukin or other polypeptide to about 200 pg/ml of interleukin or other polypeptide. In another non-limiting example, the system may detect about 90 pg/ml of interleukin or other polypeptide to about 100 pg/ml of interleukin or other polypeptide. In a non-limiting example, the system can detect at least 7,000 SARS-CoV-2 virions/μl of liquid. One of skill in the art would recognize, for example, that the system may detect from about 1000 virions/μl of SARS-CoV-2 or other virus to about 8000 virions/μl of SARS-CoV-2 or other virus. In another non-limiting example, the system may detect from about 900 virions/μl of SARS-CoV-2 or other virus to about 7000 virions/μl of SARS-CoV-2 or other virus. In another non-limiting example, the system may detect from about 800 virions/μl SARS-CoV-2 or other virus to about 6000 virions/μl SARS-CoV-2 or other virus. In another non-limiting example, the system may detect from about 700 virions/μl SARS-CoV-2 or other virus to about 5000 virions/μl SARS-CoV-2 or other virus. In another non-limiting example, the system may detect about 600 virions/μl SARS-CoV-2 or other virus to about 4000 virions/μl SARS-CoV-2 or other virus. In another non-limiting example, the system may detect from about 500 virions/μl SARS-CoV-2 or other virus to about 3000 virions/μl SARS-CoV-2 or other virus. In another non-limiting example, the system may detect about 400 virions/μl SARS-CoV-2 or other virus to about 2000 virions/μl SARS-CoV-2 or other virus. In another non-limiting example, the system may detect from about 300 virions/μl SARS-CoV-2 or other virus to about 1000 virions/μl SARS-CoV-2 or other virus. In another non-limiting example, the system may detect from about 200 virions/μl SARS-CoV-2 to about 800 virions/μl SARS-CoV-2 or other virus. In another non-limiting example, the system may detect from about 100 virions/μl SARS-CoV-2 or other virus to about 600 virions/μl SARS-CoV-2 or other virus. As larger substances have greater interference on the tapered fiber 106, SARS-CoV-2 virions are detectable at even lower concentrations (less than 7,000 SARS-CoV-2 virions/μl). Human coronavirus OC43 (HCoV-0C43) is another substance that is detectable, with the system 100 detecting it at a sensitivity as low as 50 viruses/ml. Based on these concentrations, the system can detect SARS-CoV-2 virions at clinically relevant concentrations in liquid (e.g., viral loadings that are implicated with COVID-19 infections). A resolution of 1 pg/ml is possible by optimizing the antibody surface coverage and the incubation time for the substance of interest to attach onto the antibody sites on the fiber.

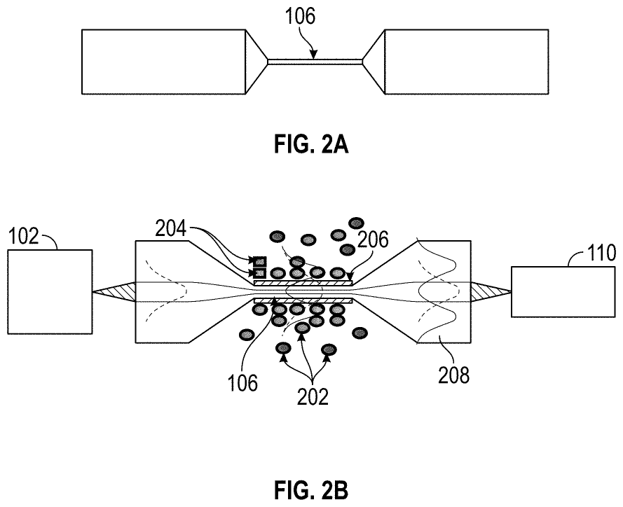

Referring to FIG. 2a, the optical fibers in tapered fiber 106 have bio-sensing capabilities by virtue of having high sensitivity. By way of an example, tapered fiber 106 is composed of a left region (L) and a right region (R) connected by a core region corresponding to functionalized surface 206, the functionalized surface being shown in FIG. 2b. In an embodiment, the lengths of the left region and the right region are the same. In another embodiment, the lengths of the left region and the right region are dissimilar. Cone-like structures are the tapering interface between R and the core region and the L and the core region. For the detection of substances, a flow of fluid around tapered fiber 106 transports target substances to functionalized surface 206. In turn, the target substances may be captured by functionalized surface 206. Light is launched from one end at fiber laser output 102 (i.e., an EMR source) of the tapered fiber 106, passes through waist region, and is collected at the other end at fiber photodetector 110. When the laser light is introduced as a single mode radiation and reaches the tapered area of tapered fiber 106, multiple cladding modes are excited simultaneously.

An electromagnetic (EM) field extends from tapered fiber 106, thereby enabling detection by detector 110 of minute changes of the refractive index close to functionalized surface 206 of tapered fiber 106. The EM field intensity exponentially decreases with distance away from functionalized surface 206 on a length scale in the order of its wavelength. EMR source 102, detector 110, and functionalized surface 206 may be modified to further boost the sensitivity in detecting the target substances. It is possible to increase the sensitivity of the system even more by reducing the diameter of the core region and increasing its length.

The transmission intensity in the output end of fiber is described as:

I_{T}=ΣₙIₙ+2Σ_{n>m}\sqrt{IₘIₙ}cos Δϕₙₘ(λ) (Eq. 1)

where the sum is over the core and cladding modes of the fiber. The first term is a constant term, but the second term produces the sinusoidal signal for the wavelength (A) that is analyzed.

The phase Δϕₙₘ in Eq. 1 is

Δϕₙₘ(λ)=(βₙ(λ)−βₘ(λ))L (Eq. 2)

where the set {βₙ(λ)} are the propagation constants of each mode in the fiber and L is the length of the waist along the direction of propagation.

If λ is scanned within a range with the fiber under different environments, there will be different outputs according to Eq. 1 and Eq. 2. The amplitude and phase of each signal are extracted from the data by Fourier analysis, where the phase change related to maximum amplitude is used. The phase shift between the two scans can be calculated to measure the change in waist of the tapered fiber 106. The phase shift may be attributed to the difference of the effective refractive index with and without binding substances.

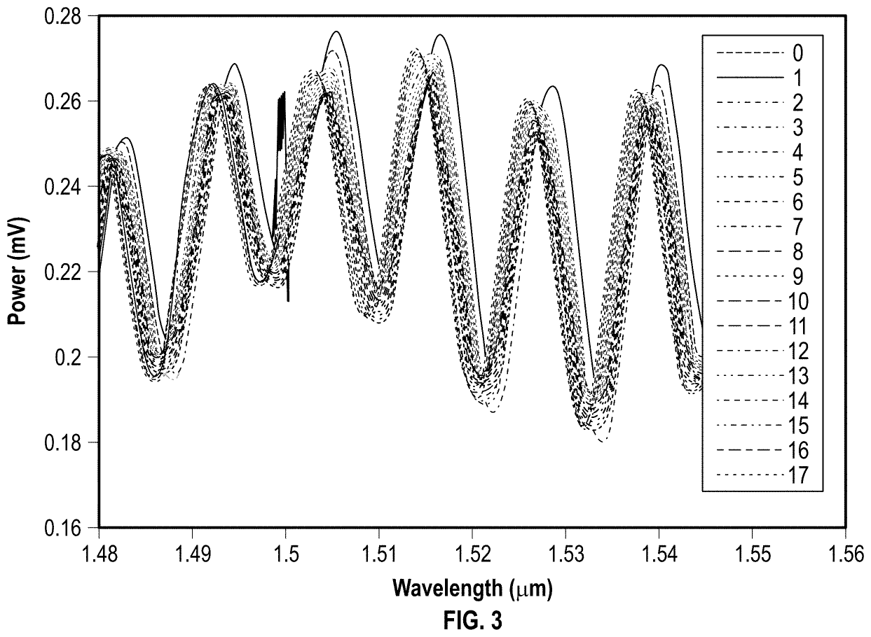

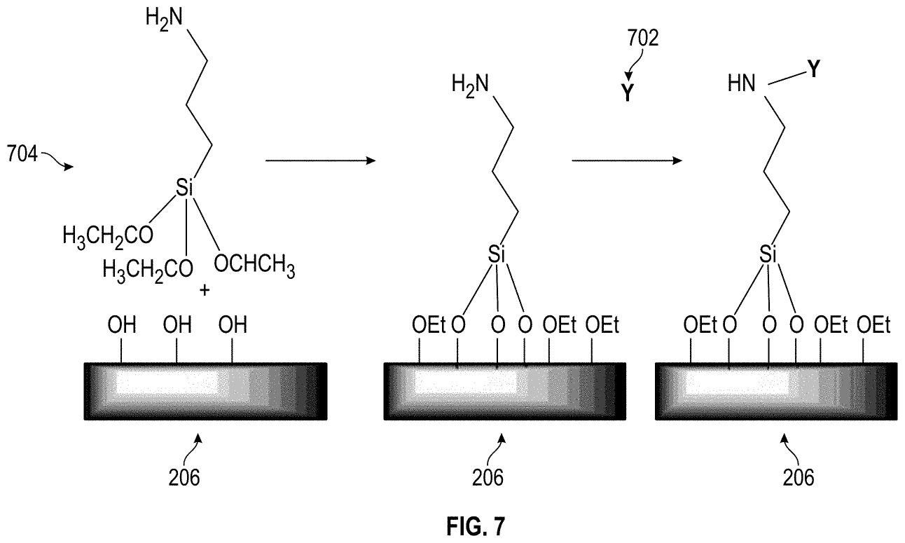

Referring to FIG. 3, the system can detect small changes in the refractive index adjacent to the tapered fiber surface. This sensing capability is attributed to the attachment of a molecular sensing element to the surface of tapered fiber 106. In an embodiment, immunoglobulin G (IgG) antibodies may be the molecular sensing element. Given that IgG molecules are approximately 28 nm in diameter, the approximate number of IgG molecules can be calculated which can be covalently tethered to tapered fiber 106. The molecular sensing element can be covalently tethered to a chemically active moiety. In an embodiment, silane chemistry introduces the molecular sensing element which attaches to the surface of tapered fiber 106 (see FIG. 7). The surface of the tapered fiber 106 reacts with the chemically active moiety amino silane, and the IgG attaches to the amino silane. In a non-limiting example, the system is able to form a binding complex where there are different concentrations of IL-8 with a phase shift in a wavelength from 1.48 μm to 1.56 μm, as depicted in FIG. 3. It is to be noted that it is possible to use other molecular sensing elements, such as aptamers, other antibodies besides IgG, and antibody fragments. In addition, difference wavelengths from the EMR source are possible. One of skill in the art would know that there are numerous chemically active moieties that can bind to the tapered fiber surface to ultimately allow for the binding of substances. Examples of such chemically active moieties include amino silanes, alkoxy silanes, epoxy silanes, vinyl silanes, methacryloxy silanes, isocyanato silanes, mercapto silanes, polysulfide silanes, ureido silanes, chromium, orthosilicate, inorganic ester, titanium, and zirconium systems.

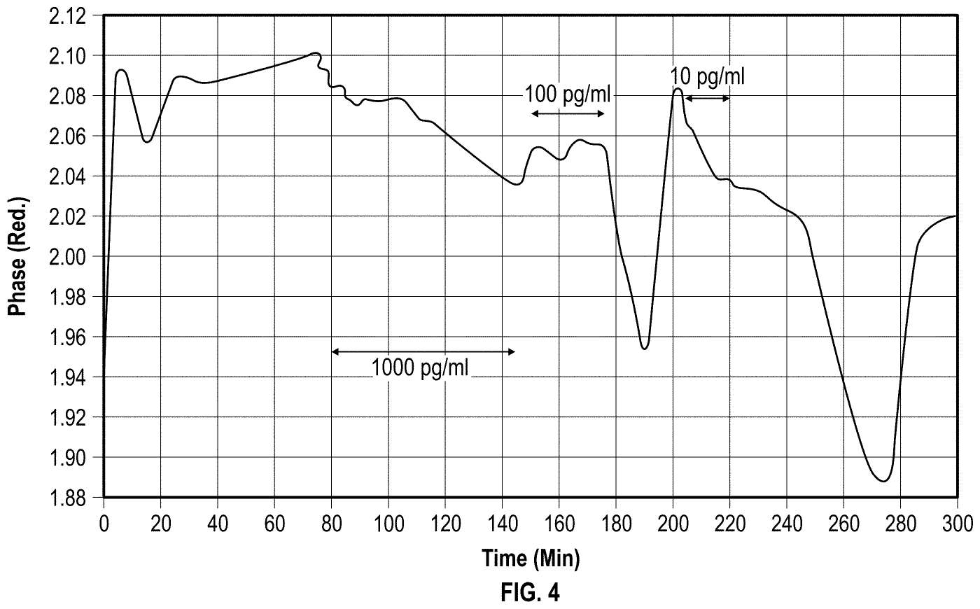

Referring to FIG. 4, phase shift is plotted against real-time. In this example, the real-time phase shift represents the binding of IL-8 to the tethered IgG (as described with respect to FIG. 3) at 3 different concentrations of IL-8: 1000 pg/ml, 100 pg/ml and 10 pg/ml. The plot in FIG. 4 is based on 65 experiments, where IL-8 antigens are detected with the system at three different concentrations of IL-8 dispersed in saliva with a pH 7.4. Noise detected by the system is around 0.007 radians. The real-time phase shift represents the binding dynamic of IL-8 to the tethered mouse anti-human IL-8 antibody (IgG). In this example, a concentration as low as 10 pg/ml or 7.1×10⁵ of IL-8/μl can be detected by the system.

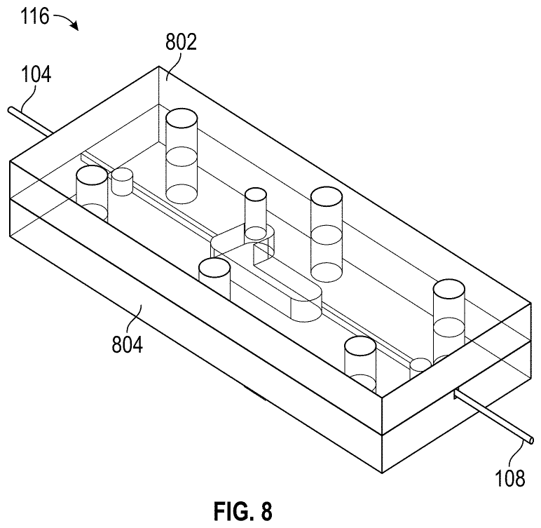

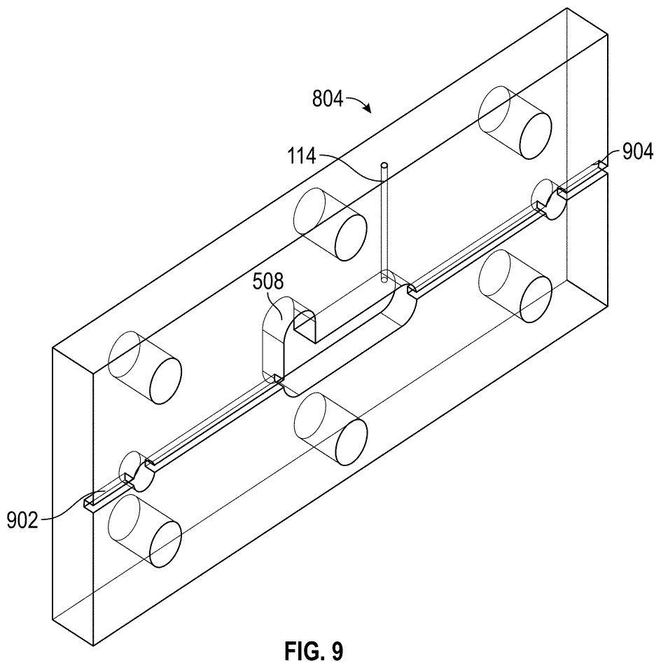

FIG. 8 depicts an embodiment of a cell 116, with a top portion 802 and a bottom portion 804. The top portion 802 and bottom portion 804 may be joined by a multitude of means, including solvent bonding, vibration welding, induction welding, mechanical fastening, soldering, brazing, riveting, diffusion bonding, glaze bonding, isotactic bonding, fusion welding, friction welding, and ultrasonic welding. The input fiber 104 allows EMR to enter the tapered fiber 106, exiting at the output fiber 108. FIG. 9 shows the reaction chamber 508 where substance and the tapered fiber 106 interact. The bottom portion 804 may have an input channel 902 and output channel 904 for the tapered fiber 106 that accommodates a tapered fiber 106. An injection tube 114 may be constructed in the bottom portion 804 to allow the substance to be introduced to the reaction chamber 508

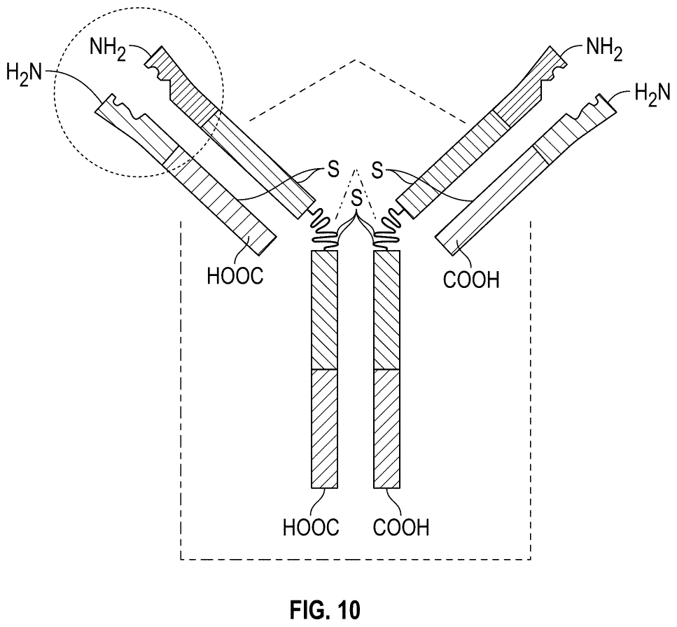

FIG. 10 is a representation of an immunoglobulin G (IgG) antibody. IgG antibodies (proteins derived from the immune systems of mammals) may be a recognition element, useful for its innate selectivity and larger relative size. IgG molecules are Y-shaped molecules composed of four amino acid chains (2 light chains, 2 heavy chains) that are joined by cysteine-cysteine disulfide bonds. The tip of the fragment (Fab) antibody region is known as the hypervariable region. The hypervariable region is the part of the molecule that is designed with a short amino acid sequence to which an antigen may bind specifically. Antigens may be foreign viruses, bacteria, proteins, cells, or even non-foreign molecules that the body creates. Given that IgG molecules are approximately 28 nm in diameter, it is possible to approximate levels of binding of IgG (or any other molecule with a known diameter) to the tapered fiber 106. With a spacing of hydroxyl groups on the surface of the tapered fiber 106 being approximately 10 groups/nm, approximately 40 fM IgG may saturate the waist region of a tapered fiber 106. With differing functionalized group spacing and molecule diameter, the molecular saturation changes. In addition to IgG, other antibodies are detectable at the femtomolar level as long as they are able to bind to the tapered fiber 106.

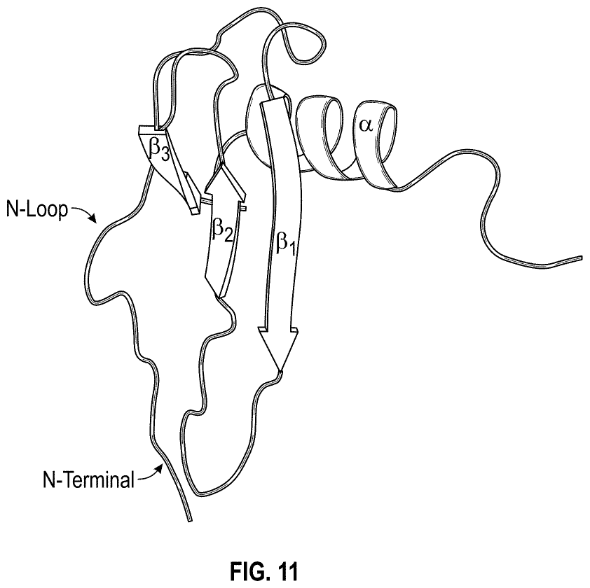

FIG. 11 is a representation of IL-8, a well-studied chemokine. The structure of IL-8 comprises a short N-terminal region, an extended N-loop region, followed by three β-strands and an α-helix. Structure-function studies indicate N-terminal and N-loop residues bind to IL-8 antibody.

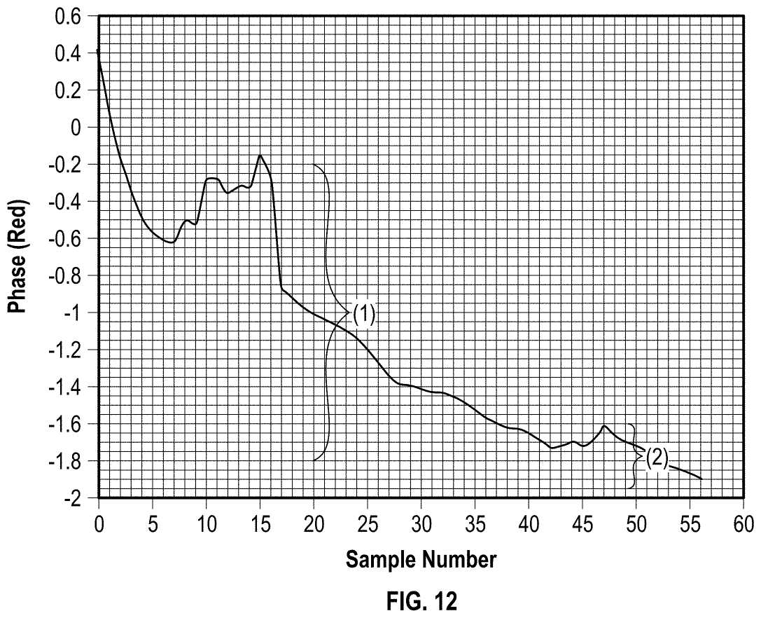

FIG. 12 is a graph that depicts the phase change associated with adding antigen to a cell. Various fluids may be used to introduce the substance (in this instance an antigen) to the system. In this instance, the fluid is phosphate-buffered saline (PBS). The tapered fiber is coated by antibody in which the antigen may attach. Antigen was then added to the cell at points (1) and (2), with phase change being determined after each addition of antigen. As time elapses for more antigen to be able to attach to the functionalized surface of the tapered fiber, the phase change is significant, as shown in (1). With (2), the phase change is smaller, as there is likely less opportunity for antigen to bind to the antibodies. Furthermore, the signal-to-noise-ratio (SNR) can be calculated by noting the amount of change in the phase between the time before the first and the second phase change, with an estimate of the noise stemming from the standard deviation of the phase change fluctuations. A SNR of 10 is found, which is sufficiently adequate for signal measurement. The system 100 provides repeatable results, in that it is possible to strip substance from the tapered fiber 106 once testing is complete. In an embodiment, a glycine solution at pH 2.4 is used to strip substance from the tapered fiber 106. In the same embodiment, once glycine is removed another fluid may be added to the cell 116 to flush the cell 116 and return the cell 116 to a neutral pH. Incubation time for the glycine solution to interact with tapered fiber varies. In an embodiment, the glycine solution is incubated with the tapered fiber 106 for between 15 minutes and 25 minutes. In another embodiment, the glycine solution is incubated with the tapered fiber 106 for between 5 minutes and 30 minutes. In yet another embodiment, the glycine solution is incubated with the tapered fiber 106 for between 1 minute and 30 minutes. For the signal to be detectable, a certain SNR is desired. In an embodiment, the SNR may be at least 2. In another embodiment, the SNR may range from 2 to 10. In another embodiment, the SNR may be greater than 10.

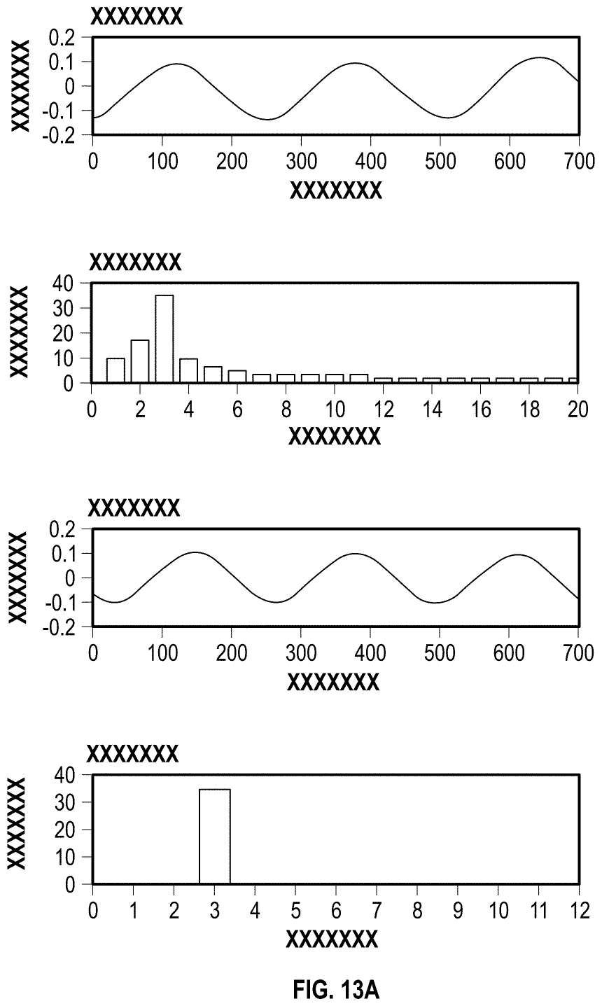

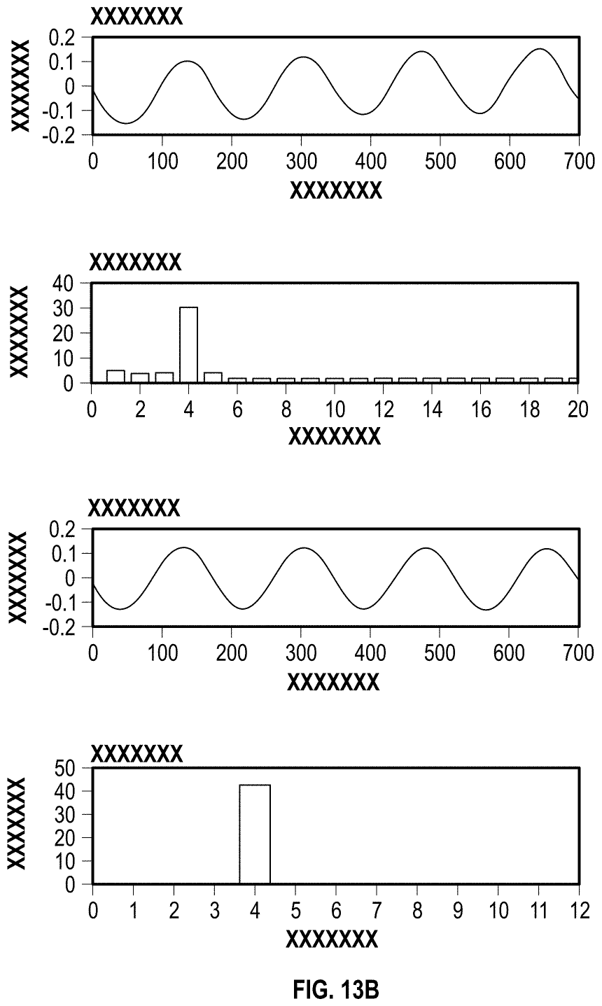

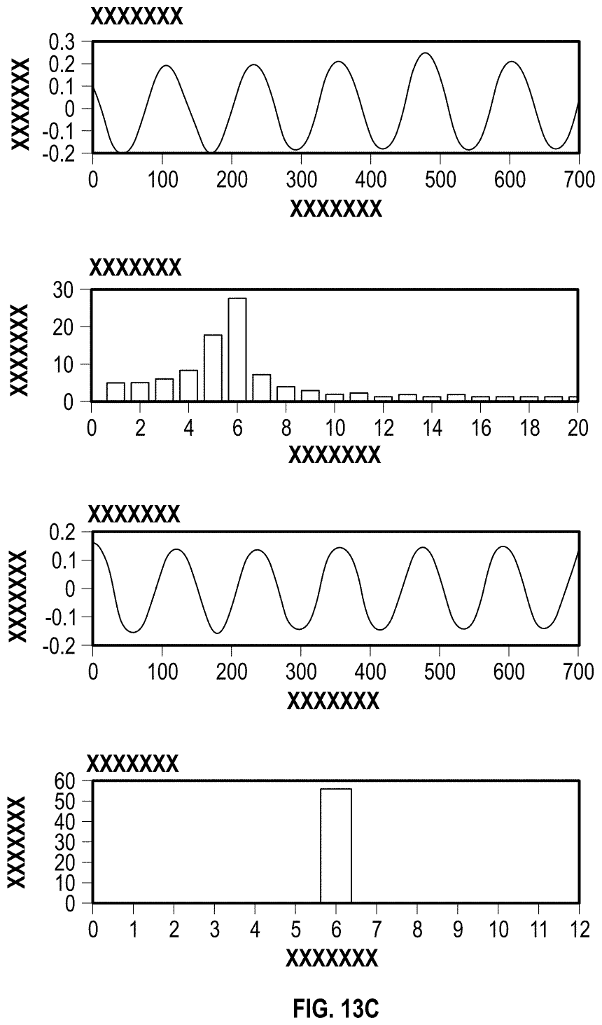

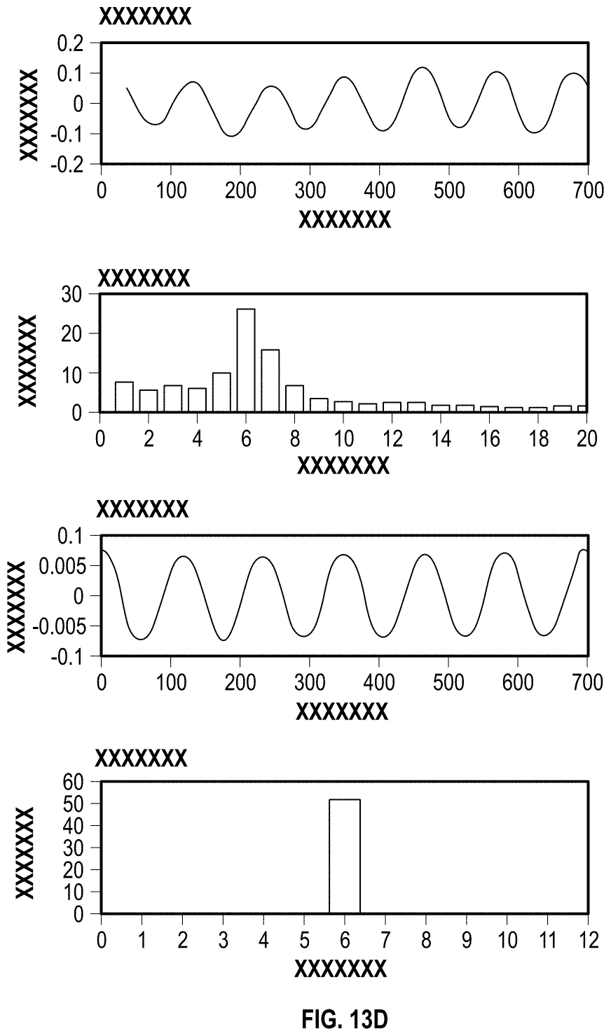

FIGS. 13a, 13b, 13c, and 13d are representative graphs of signals from cells with tapered fiber lengths of 4 mm, 6 mm, 8 mm, and 10 mm, respectively. In this instance, the diameter of the fiber is 10 microns with water as the fluid. We have experimentally confirmed that a sensing length of 6 mm and using a tapered fiber diameter of 10 μm offers optimal design with the lowest background noise, resulting in stable and repeatable results. In contrast, there are more frequency noise for tapered diameter 12 μm. An even larger tapered length may yield a higher dominant frequency (DF), which enhances the sensitivity of the system. Sensitivity of the system 100 may also be affected by fiber stress, liquid flow, and room temperature.

### EXAMPLES

Aspects of the present teachings may be further understood in light of the following examples, which should not be construed as limiting the scope of the present teachings in any way.

### Example 1—System in Portable Form

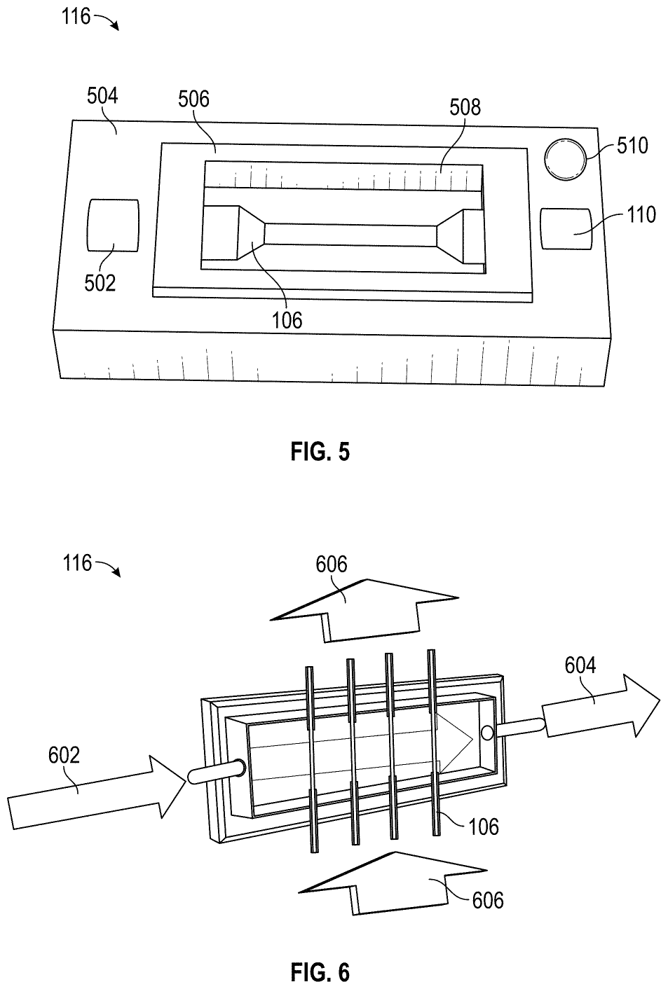

In an embodiment of the system 100, the EMR wavelength continuously tunes over a spectral band from 1.48 to 1.56 μm. In an embodiment, the spectral band is from 0.4 microns to 0.6 micron. In another embodiment, the spectral band is from 0.6 to 0.8 microns. In another embodiment, the spectral band is from 0.8 to 1 microns. In another embodiment, the spectral band is from 1 to 1.5 microns. In another embodiment, the spectral band is from 1.5 to 2 microns. In another embodiment, the spectral band from 2 microns to 3.5 microns. The precise wavelength range is not critical. The light is launched into tapered fiber 106 and passes to tapered fiber 106 inside reaction chamber 508. Then the light transmitted through the tapered region of tapered fiber 106 enters another fiber segment of tapered fiber 106 connected to photodetector 110. In FIG. 1, the EMR source 102 and detector 110 of the system are part of a same machine. However, the tunable laser may be bulky and expensive. Therefore, as seen in FIG. 5 a low-cost small laser diode 502 may be used to replace the tunable laser. Similar to LEDs, laser diode 502 is a diode pumped directly with electrical current for creating lasing conditions at the diode's junction. LED indicator 510 is activated (i.e., lights up) when substances bind to the molecular sensing element, which is attached to the chemically active moiety, which is connected to the surface of tapered fiber 106 (i.e., the binding complex). Semiconductor material determines the wavelength of the emitted light range from infrared (IR) to the ultraviolet (UV) spectrum. The detector in FIG. 1 can be replaced with a small inexpensive fiber photodetector 110, as depicted in FIG. 5. Additionally, cartridge 506 is disposable. Cartridge 506 can be inserted into reusable reading module 504, such that cartridge 506 surrounds reaction chamber 508. In an embodiment, the cell 116 may be placed in patient's mouth to concentration of a substance in saliva. In another embodiment, the cell 116 may be on a benchtop.

Referring to FIG. 6, in an embodiment, there are multiple tapered fibers 106, housed in the same cell. Each tapered fiber 106 binds a different substance. The phase shift of EMR will differ based on the substance bound to each tapered fiber 106. In such an arrangement, it is possible to detect multiple conditions based on the substances bound to the taper fibers 106. Substance to be detected flows into the cell through the flow in channel 602, exiting in the flow out channel 604. EMR enters the tapered fibers 106 via the EMR input 606 and exits from the EMR output 608.

### Example 2—Label-Free Biomolecule Detection

To prepare label-free antigen detection, as an example, APTES (3-aminopropyl triethoxysilane), the chemically active moiety 704, as seen in FIG. 7, is bound on the surface of tapered fiber 106, wherein the surface has hydroxyl groups. The silane terminal amino group is used to covalently tether the molecular sensing element 702, an antibody in this embodiment, to the fiber surface, as depicted in FIG. 7. The systems and methods herein use two scenarios for testing the system: (1) a generic antibody-antigen pair; and (2) an IL-8 specific antibody for detection of antigen IL-8. Those procedures have been standardized and routinely performed.

Epstein-Barr virus (EBV) in saliva can be subsequently investigated by the system as a surrogate virus for SARS-CoV-2. In the United States, approximately 50% of children and 90% of adults have been infected with EBV and gained adaptive immunity without any symptoms. Infection with EBV occurs by the oral transfer of saliva. While EBV is a DNA virus and SARS-CoV-2 is a RNA virus, EBV and SARS-CoV-2 are: (i) similar in size (˜120 nm) and (ii) have a viral envelope with protein receptors for binding and entering human cells. The low pathogenicity, high prevalence, and saliva transmission make EBV a suitable surrogate for label-free direct detection of SARS-CoV-2.

Fifty adults are recruited for this study, where ˜5 people (10%) are negative for EBV infection. Both blood and saliva are collected from all participants. ELISA assays determine EBV positive or negative individuals (Using Diamedix™ Immunosimplicity™ EBV-VCA IgG Test Kit and Arlington Scientific EBV-VCA IgG ELISA Test Kit, Fisher Scientific, USA). Viral titers in saliva of infected people are determined by PCR (artus EBV PCR Kits C E, Qiagen, USA). Anti-EBV gp340/gp220 Envelope Antibody (sc-57724, Santa Cruz Biotechnology, USA) is tethered to a tapered fiber as described in FIG. 5. Saliva (500 μl) is processed with a microfluidic filter designed with minimal dead volume for filtering EBV virions. A filter membrane of 200 nm pores allows virions to pass, while removing cellular debris. The resulting saliva in a syringe is subjected to another filter with 80 nm pores. The first 100 μl of saliva passed through the filter (without virus as a control input) is added to the reaction chamber for background signal calibration. After removing the control input from chamber and the filter from syringe, another 100 μl of saliva (from the same sample) with virions are added for binding to the tapered fiber (5 minutes). The phase shift of light is calculated and processed on reading module 20.

### Example 3—Using the System

The system can be used for detecting substances implicated in many conditions. Example 2 highlighted one such condition (EBV for detecting mononucleosis). Based on the properties of the tapered surface (waist diameter and length), the system obviates label-based testing. While not an exhaustive list, the system can detect the following physiological conditions: mononucleosis, Anthrax infection, thyroid cancer, prostate cancer, lung cancer, esophageal cancer, congestive heart failure, myocardial infarction, diabetes, glucose anomalies, Parkinson's Disease, Alzheimer's Disease, Tuberculosis, and the common cold. Essentially, any condition that is associated with a substance that can bind to the taper fiber in the system is detectable. Substances that are detectable by the system include SARS-CoV-1, SARS-CoV-2, HPV, FSH, LH, and other substances found in body fluid, which are disclosed herein and known to those of skill in the art.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which does not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Specifically intended to be within the scope of the present invention, and incorporated herein by reference in its entirety, is the following publications:
Wyllie A L, Fournier J, Casanovas-Massana A, et al. Saliva is more sensitive for SARS-CoV-2 detection in COVID-19 patients than nasopharyngeal swabs. medRxiv. 2020:2020.2004.2016.20067835.
Crowe D L, Hacia J G, Hsieh C L, Sinha U K, Rice H. Molecular pathology of head and neck cancer. Histol Histopathol. 2002; 17(3):909-914.
Sinha U K, Ng M. Surgery of the salivary glands. Otolaryngol Clin North Am. 1999; 32(5):887-906.
Korostoff A, Reder L, Masood R, Sinha U K. The role of salivary cytokine biomarkers in tongue cancer invasion and mortality. Oral Oncol. 2011; 47(4):282-287.
Zhong J F, Weiner L P, Burke K, Taylor C R. Viral RNA extraction for in-the-field analysis. J Virol Methods. 2007; 144 (1-2):98-102.
Zhong J F, Weiner L, Jin Y, Lu W, Taylor C R. A real-time pluripotency reporter for human stem cells. Stem Cells Dev. 2010; 19(1):47-52.
Khan A, Liu Q, Chen X, et al. Detection of human papillomavirus in cases of head and neck squamous cell carcinoma by RNA-seq and VirTect. Mol Oncol. 2019; 13(4):829-839.
Zhong J F, Chen Y, Marcus J S, et al. A microfluidic processor for gene expression profiling of single human embryonic stem cells. Lab Chip. 2008; 8(1):68-74.
Chen Y, Zhang B, Feng H, Shu W, Chen G Y, Zhong J F. An automated microfluidic device for assessment of mammalian cell genetic stability. Lab Chip. 2012; 12(20):3930-3935.
Fan J B, Chen J, April C S, et al. Highly parallel genome-wide expression analysis of single mammalian cells. PLoS One. 2012; 7 (2):e30794.
Li Z, Zhang C, Weiner L P, Zhang Y, Zhong J F. Molecular characterization of heterogeneous mesenchymal stem cells with single-cell transcriptomes. Biotechnol Adv. 2013; 31(2):312-317.
Chen Y, Millstein J, Liu Y, et al. Single-Cell Digital Lysates Generated by Phase-Switch Microfluidic Device Reveal Transcriptome Perturbation of Cell Cycle. ACS Nano. 2018; 12(5):4687-4694.
Wang L, Wang Y, Ye D, Liu Q. Review of the 2019 novel coronavirus (SARS-CoV-2) based on current evidence. Int J Antimicrob Agents. 2020:105948.
Baldwin E T, Weber I T, St Charles R, et al. Crystal structure of interleukin 8: symbiosis of NMR and crystallography. Proc Natl Acad Sci USA. 1991; 88(2):502-506.
Clore G M, Appella E, Yamada M, Matsushima K, Gronenborn A M. Three-dimensional structure of interleukin 8 in solution. Biochemistry. 1990; 29(7):1689-1696.
Mowbray S E, Amiri A M. A Brief Overview of Medical Fiber Optic Biosensors and Techniques in the Modification for Enhanced Sensing Ability. 2019; 9 (1).
Korposh S, James S W, Lee S W, Tatam R P. Tapered Optical Fibre Sensors: Current Trends and Future Perspectives. Sensors (Basel). 2019; 19 (10).
Garcia Mina D, Haus J W, Chong A, Khanolkar A, Sarangan A, Hansen K. Bi-tapered fiber sensor using visible to near infrared light. Sensors and Actuators A: Physical. 2017; 263:285-290.
Jauregui-Vazquez D, Haus J W, Negari A B H, Sierra-Hernandez J M, Hansen K. Bitapered fiber sensor: Signal analysis. Sensors and Actuators B: Chemical. 2015; 218:105-110.
Herrera Piad L A, Haus J, Jauregui-Vazquez D, et al. Magnetic Field Sensing Based on Bi-Tapered Optical Fibers Using Spectral Phase Analysis. Sensors. 2017; 17:2393.
King B, Idehenre I, Powers P, Sarangan A, Haus J, Hansen K. Tapered optical fibers for aqueous and gaseous phase biosensing applications. Vol. 8570; 2013.
Amin Z. Diode lasers: Experimental and clinical review. Lasers in Medical Science. 1995; 10(3):157-163.
Kerr J R. Epstein-Barr virus (EBV) reactivation and therapeutic inhibitors. J Clin Pathol. 2019; 72(10):651-658.
Buschle A, Hammerschmidt W. Epigenetic lifestyle of Epstein-Barr virus. 2020; 42(2):131-142.
Odumade O A, Hogquist K A, Balfour H H, Jr. Progress and problems in understanding and managing primary Epstein-Barr virus infections. Clin Microbiol Rev. 2011; 24(1):193-209.

What is claimed is:

1. A system for detecting a substance in a fluid, the system comprising:
a. an electromagnetic radiation source;
b. an input fiber connected to the electromagnetic radiation source;
c. at least one tapered fiber connected to the input fiber with the substance bound to the at least one tapered fiber;
d. an output fiber connected to the at least one tapered fiber; and
e. a photodetector connected to the output fiber.

2. The system of claim 1, wherein the electromagnetic radiation source is selected from the group consisting of laser-emitting diode, laser diode, pulse wave laser, continuous wave laser, and tunable laser.

3. The system of claims 1 and 2, further comprising an auto-injector connected to the at least one tapered fiber.

4. The system of claims 1 through 3, wherein the at least one tapered fiber is housed in a cell.

5. The system of claims 1 through 4, wherein the substance is selected from the group consisting of biomolecules, analytes, biomarkers, fungus, electrolyes, hormones, bacteria, viruses, parasites, proteins, organic molecules, volatile organic compounds, and nucleotides.

6. The system of claims 1 through 5, wherein the fluid is selected from the group consisting of cerebrospinal fluid, drain fluid, saliva, urine, blood, plasma, tears, bronchioalveolar lavage fluid, serum, pleural fluid, synovial fluid, peritoneal fluid, amniotic fluid, gastric fluid, lymph fluid, interstitial fluid, tissue homogenate, cell extracts, sputum, stool, physiological secretions, mucus, sweat, milk, semen, seminal fluid, vaginal secretions, fluid from ulcers and surface eruptions, blisters, and abscesses, forever chemicals in water, perfluorooctanoic acid in water, perfluorooctanesulfonic acid in water, and extracts of tissues including biopsies of normal, malignant, and suspect tissues, and exhaled breath condensate.

7. The system of claims 1 through 6, wherein the at least one tapered fiber binds more than one substance.

8. The system of claims 1 through 7, wherein the at least one tapered fiber is bound with a chemically active moiety.

9. The system of claims 1 through 8, wherein a molecular sensing element binds to the chemically active moiety.

10. The system of claims 1 through 9, wherein the chemically active moiety is selected from the group consisting of amino silane, alkoxy silanes, epoxy silanes, vinyl silanes, methacryloxy silanes, isocyanato silanes, mercapto silanes, polysulfide silanes, ureido silanes, chromium, orthosilicate, inorganic ester, titanium, and zirconium systems.

11. The system of claims 1 through 10, wherein the molecule sensing element is a binding molecule.

12. A method of detecting a substance, the method comprising:
a. binding the substance to an at least one tapered fiber;
b. passing electromagnetic radiation through the at least one tapered fiber from an electromagnetic radiation source; and
c. generating a value from a photodetector based on a phase shift of the electromagnetic radiation caused by the binding of the substance to the at least one tapered fiber.

13. The method of claim 12, wherein the substance is in a fluid.

14. The method of claims 12 and 13, wherein the at least one tapered fiber is housed in a cell.

15. The method of claims 12 through 14, wherein the electromagnetic radiation source is selected from the group consisting of light emitting diode, laser diode, pulse wave laser, continuous wave laser, and tunable laser.

16. A method for detecting a physiological condition in a subject, the method comprising:
a. binding a substance in a fluid of the subject to an at least one tapered fiber;
b. passing light through the at least one tapered fiber from an electromagnetic radiation source;
c. generating a value from a photodetector based on a phase shift of the light caused by the binding of the substance to the at least one tapered fiber; and
d. assigning a score to the value, with the score indicating a likelihood of the subject having the physiological condition.

17. The method of claim 16, wherein if the score is closer to zero, the likelihood is less likely to have the physiological condition, and if the score is further from zero, the likelihood is more likely to have the physiological condition.

18. The method of claims 16 and 17, wherein the physiological condition is selected from the group consisting of COVID-19, mononucleosis, Anthrax, thyroid cancer, prostate cancer, lung cancer, esophageal cancer, congestive heart failure, myocardial infarction, diabetes, glucose anomalies, Parkinson's Disease, Alzheimer's Disease, Tuberculosis, pregnancy, and common cold.

19. The method of claims 16 through 18, wherein the score is compared to an assessment by a medical professional to determine false positives and false negatives.

20. The method of claims 16 through 19, wherein the score is changeable based on changes to the substance bound to the at least one tapered fiber.
